(19) United States
(12) Patent Application Publication
(10) Pub. No.: US 2024/0307875 A1
(43) Pub. Date: Sep. 19, 2024

(54) MICROFLUIDIC CHIP AND LAMP GENE AMPLIFICATION METHOD

(71) Applicant: UIF (University Industry Foundation), Yonsei University, Seoul (KR)

(72) Inventors: Hyo Il JUNG, Seoul (KR); Cheng NIE, Seoul (KR)

(73) Assignee: UIF (University Industry Foundation), Yonsei University, Seoul (KR)

(21) Appl. No.: 18/410,669

(22) Filed: Jan. 11, 2024

(30) Foreign Application Priority Data
Mar. 17, 2023 (KR) 10-2023-0035151

(51) Int. Cl. B01L 3/00; C12Q 1/6844

(52) U.S. Cl. CPC B01L 3/502753; C12Q 1/6846; B01L 2300/0883; B01L 2400/086

(57) ABSTRACT

A microfluidic chip and a LAMP gene amplification method using the same are disclosed. The microfluidic chip includes a fluid inlet; a fluid outlet; and a flow channel constructed to connect the fluid inlet and the fluid outlet to each other, wherein a fluid flows in the flow channel, wherein the flow channel includes a plurality of eddy generation structures connected in series with each other, wherein each of the eddy generation structures includes: a main flow channel; and at least one auxiliary flow channel branching from the main flow channel at a first point thereof, and merging with the main flow channel at a second point thereof spaced apart from the first point in a fluid flow direction, wherein the auxiliary flow channel meets with the main flow channel at an angle in a range of 90° exclusive to 180° exclusive.

### CROSS-REFERENCE TO RELATED APPLICATION

This application claims a benefit under 35 U.S.C. § 119(a) of Korean Patent Application No. 10-2023-0035151 filed on Mar. 17, 2023, with the Korean Intellectual Property Office, the entire disclosure of which is incorporated herein by reference for all purposes.

### BACKGROUND

### 1. Field

The present disclosure relates to a microfluidic chip and a LAMP gene amplification method.

### 2. Description of Related Art

Among various nucleic acid amplification technologies (NAAT), loop-mediated isothermal amplification (LAMP) has the potential to be developed in a point-of-care test because it operates isothermally within a short test time. However, the LAMP process is required to be automated for field use because it requires a series of steps such as pipetting to mix and react various reagents with each other.

### SUMMARY

A purpose of the present disclosure is to provide a microfluidic chip to allow the LAMP process to be automated.

Another purpose of the present disclosure is to provide a LAMP gene amplification method using the microfluidic chip.

Purposes according to the present disclosure are not limited to the above-mentioned purpose. Other purposes and advantages according to the present disclosure that are not mentioned may be understood based on following descriptions, and may be more clearly understood based on embodiments according to the present disclosure. Further, it will be easily understood that the purposes and advantages according to the present disclosure may be realized using means shown in the claims or combinations thereof.

A first aspect of the present disclosure provides a microfluidic chip comprising: a fluid inlet; a fluid outlet; and a flow channel constructed to connect the fluid inlet and the fluid outlet to each other, wherein a fluid flows in the flow channel, wherein the flow channel includes a plurality of eddy generation structures connected in series with each other, wherein each of the eddy generation structures includes: a main flow channel; and at least one auxiliary flow channel branching from the main flow channel at a first point thereof, and merging with the main flow channel at a second point thereof spaced apart from the first point in a fluid flow direction, wherein the auxiliary flow channel meets with the main flow channel at an angle in a range of 90° exclusive to 180° exclusive.

In accordance with some embodiments of the microfluidic chip, a loop-mediated isothermal amplification (LAMP) reaction is carried out within the flow channel.

In accordance with some embodiments of the microfluidic chip, the auxiliary flow channel of the eddy generation structure meets with the main flow channel at an angle of 130 to 140°.

In accordance with some embodiments of the microfluidic chip, the main flow channel has a width of 80 to 120 μm.

In accordance with some embodiments of the microfluidic chip, an auxiliary flow channel includes: a first portion branching from the first point of the main flow channel and extending by a first length; a second portion extending in a curved manner from an end of the first portion toward the main flow channel; and a third portion extending from an end of the second portion by a second length and merging with the second point of the main flow channel.

In accordance with some embodiments of the microfluidic chip, the at least one auxiliary flow channel includes a plurality of auxiliary flow channels disposed on both opposing sides of the main flow channel and arranged in a zigzag manner along the main flow channel.

In accordance with some embodiments of the microfluidic chip, the main flow channel is formed in a serpentine structure including a plurality of rows.

In accordance with some embodiments of the microfluidic chip, the fluid inlet includes a plurality of the fluid inlets.

A second aspect of the present disclosure provides a LAMP gene amplification method comprising: providing a microfluidic chip, wherein the microfluidic chip includes: a fluid inlet; a fluid outlet; and a flow channel constructed to connect the fluid inlet and the fluid outlet to each other, wherein a fluid flows in the flow channel, wherein the flow channel includes a plurality of eddy generation structures connected in series with each other, wherein each of the eddy generation structures includes: a main flow channel; and at least one auxiliary flow channel branching from the main flow channel at a first point thereof, and merging with the main flow channel at a second point thereof spaced apart from the first point in a fluid flow direction, wherein the auxiliary flow channel meets with the main flow channel at an angle in a range of 90° exclusive to 180° exclusive; and injecting a LAMP reactant into the fluid inlet such that the LAMP reactant flows along the flow channel.

In accordance with some embodiments of the method, the auxiliary flow channel of the eddy generation structure meets with the main flow channel at an angle of 130 to 140°.

In accordance with some embodiments of the method, the main flow channel has a width of 80 to 120 μm.

In accordance with some embodiments of the method, an auxiliary flow channel includes: a first portion branching from the first point of the main flow channel and extending by a first length; a second portion extending in a curved manner from an end of the first portion toward the main flow channel; and a third portion extending from an end of the second portion by a second length and merging with the second point of the main flow channel.

In accordance with some embodiments of the method, the at least one auxiliary flow channel includes a plurality of auxiliary flow channels disposed on both opposing sides of the main flow channel and arranged in a zigzag manner along the main flow channel.

In accordance with some embodiments of the method, the main flow channel is formed in a serpentine structure including a plurality of rows.

In accordance with some embodiments of the method, the fluid inlet includes a plurality of the fluid inlets.

The microfluidic chip according to an embodiment of the present disclosure has the eddy generation structure similar to an inverted Tesla structure, thereby enabling automation of the LAMP reaction.

The LAMP gene amplification method according to an embodiment of the present disclosure may allow the LAMP reaction to be automated using the microfluidic chip.

Effects of the present disclosure are not limited to the effects mentioned above, and other effects not mentioned will be clearly understood by those skilled in the art from the descriptions below.

### BRIEF DESCRIPTION OF DRAWINGS

FIG. 1 is a diagram showing a microfluidic chip according to an embodiment of the present disclosure.

FIG. 2 is a flowchart showing a LAMP gene amplification method according to an embodiment of the present disclosure.

### DETAILED DESCRIPTIONS

Hereinafter, embodiments of the present disclosure will be described in detail with reference to the accompanying drawings. The present disclosure may have various changes and may have various forms. Thus, specific embodiments are illustrated in the drawings and described in detail herein. However, this is not intended to limit the present disclosure to a specific disclosure form. It should be understood that the present disclosure includes all changes, equivalents, or substitutes as included within the spirit and technical scope of the present disclosure. The embodiments are set forth only to make the present disclosure complete, and to entirely inform the scope of the present disclosure to those of ordinary skill in the technical field to which the present disclosure belongs, and the present disclosure is only defined by the scope of the claims.

For simplicity and clarity of illustration, elements in the drawings are not necessarily drawn to scale. The same reference numbers in different drawings represent the same or similar elements, and as such perform similar functionality. Further, descriptions and details of well-known steps and elements are omitted for simplicity of the description. Furthermore, in the following detailed description of the present disclosure, numerous specific details are set forth in order to provide a thorough understanding of the present disclosure. However, it will be understood that the present disclosure may be practiced without these specific details. In other instances, well-known methods, procedures, components, and circuits have not been described in detail so as not to unnecessarily obscure aspects of the present disclosure. Examples of various embodiments are illustrated and described further below. It will be understood that the description herein is not intended to limit the claims to the specific embodiments described. On the contrary, it is intended to cover alternatives, modifications, and equivalents as may be included within the spirit and scope of the present disclosure as defined by the appended claims.

A shape, a size, a ratio, an angle, a number, etc. disclosed in the drawings for illustrating embodiments of the present disclosure are illustrative, and an embodiment of embodiments of the present disclosure are not limited thereto.

The terminology used herein is directed to the purpose of describing particular embodiments only and is not intended to be limiting of the present disclosure. As used herein, the singular constitutes “a” and “an” are intended to include the plural constitutes as well, unless the context clearly indicates otherwise. It will be further understood that the terms “comprise”, “comprising”, “include”, and “including” when used in this specification, specify the presence of the stated features, integers, operations, elements, and/or components, but do not preclude the presence or addition of one or more other features, integers, operations, elements, components, and/or portions thereof. As used herein, the term “and/or” includes any and all combinations of one or more of associated listed items. Expression such as “at least one of” when preceding a list of elements may modify the entire list of elements and may not modify the individual elements of the list. In interpretation of numerical values, an error or tolerance therein may occur even when there is no explicit description thereof.

It will also be understood that when a first element or layer is referred to as being present “on” a second element or layer, the first element may be disposed directly on the second element or may be disposed indirectly on the second element with a third element or layer being disposed between the first and second elements or layers. It will also be understood that when a first element or layer is referred to as being present “under” a second element or layer, the first element may be disposed directly under the second element or may be disposed indirectly under the second element with a third element or layer being disposed between the first and second elements or layers.

It will be understood that when an element or layer is referred to as being “connected to”, or “coupled to” another element or layer, it may be directly connected to or coupled to another element or layer, or one or more intervening elements or layers therebetween may be present. In addition, it will also be understood that when an element or layer is referred to as being “between” two elements or layers, it may be the only element or layer between the two elements or layers, or one or more intervening elements or layers therebetween may also be present.

In addition, it will also be understood that when an element or layer is referred to as being “between” two elements or layers, it may be the only element or layer between the two elements or layers, or one or more intervening elements or layers therebetween may also be present.

Further, as used herein, when a layer, film, region, plate, or the like is disposed “on” or “on a top” of another layer, film, region, plate, or the like, the former may directly contact the latter or still another layer, film, region, plate, or the like may be disposed between the former and the latter. As used herein, when a layer, film, region, plate, or the like is directly disposed “on” or “on a top” of another layer, film, region, plate, or the like, the former directly contacts the latter and still another layer, film, region, plate, or the like is not disposed between the former and the latter. Further, as used herein, when a layer, film, region, plate, or the like is disposed “below” or “under” another layer, film, region, plate, or the like, the former may directly contact the latter or still another layer, film, region, plate, or the like may be disposed between the former and the latter. As used herein, when a layer, film, region, plate, or the like is directly disposed “below” or “under” another layer, film, region, plate, or the like, the former directly contacts the latter and still another layer, film, region, plate, or the like is not disposed between the former and the latter.

When a certain embodiment may be implemented differently, a function or an operation specified in a specific block may occur in a different order from an order specified in a flowchart. For example, two blocks in succession may be actually performed substantially concurrently, or the two blocks may be performed in a reverse order depending on a function or operation involved.

It will be understood that, although the terms “first”, “second”, “third”, and so on may be used herein to describe various elements, components, regions, layers and/or sections, these elements, components, regions, layers and/or sections should not be limited by these terms. These terms are used to distinguish one element, component, region, layer or section from another element, component, region, layer or section. Thus, a first element, component, region, layer or section described under could be termed a second element, component, region, layer or section, without departing from the spirit and scope of the present disclosure.

Spatially relative terms, such as “beneath,” “below,” “lower,” “under,” “above,” “upper,” and the like, may be used herein for ease of illustration to illustrate one element or feature's relationship to another element or feature as illustrated in the figures. It will be understood that the spatially relative terms are intended to encompass different orientations of the device in use or in operation, in addition to the orientation depicted in the figures. For example, when the device in the drawings may be turned over, elements described as “below” or “beneath” or “under” other elements or features would then be oriented “above” the other elements or features. Thus, the example terms “below” and “under” may encompass both an orientation of above and below. The device may be otherwise oriented, for example, rotated 90 degrees or at other orientations, and the spatially relative descriptors used herein should be interpreted accordingly.

The features of the various embodiments of the present disclosure may be partially or entirely combined with each other, and may be technically associated with each other or operate with each other. The embodiments may be implemented independently of each other and may be implemented together in an association relationship.

In interpreting a numerical value, the value is interpreted as including an error range unless there is no separate explicit description thereof.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As used herein, “embodiments,” “examples,” “aspects, and the like should not be construed such that any aspect or design as described is superior to or advantageous over other aspects or designs.

Further, the term ‘or’ means ‘inclusive or’ rather than ‘exclusive or’. That is, unless otherwise stated or clear from the context, the expression that ‘x uses a or b’ means any one of natural inclusive permutations.

The terms used in the description below have been selected as being general and universal in the related technical field. However, there may be other terms than the terms depending on the development and/or change of technology, convention, preference of technicians, etc. Therefore, the terms used in the description below should not be understood as limiting technical ideas, but should be understood as examples of the terms for illustrating embodiments.

Further, in a specific case, a term may be arbitrarily selected by the applicant, and in this case, the detailed meaning thereof will be described in a corresponding description section. Therefore, the terms used in the description below should be understood based on not simply the name of the terms, but the meaning of the terms and the contents throughout the Detailed Descriptions.

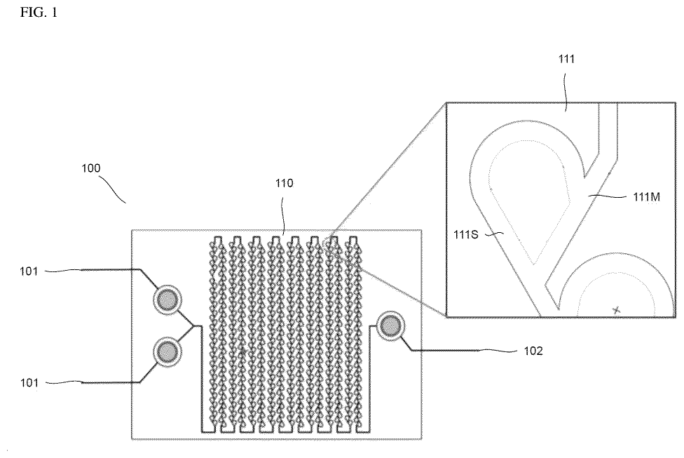

FIG. 1 is a diagram showing a microfluidic chip according to an embodiment of the present disclosure.

Referring to FIG. 1, a microfluidic chip 100 according to an embodiment of the present disclosure includes a fluid inlet 101; a fluid outlet 102; and a flow channel 110 that connects the fluid inlet 101 and the fluid outlet 102 to each other and allows fluid to flow therein, wherein the flow channel 110 includes a plurality of eddy generation structures 111 connected in series with each other, and each of the eddy generation structures 111 includes a main flow channel 111M, and one or more auxiliary flow channels 111S branched from the main flow channel 111M at a first point thereof, and merges with the main flow channel 111M at a second point thereof spaced apart from the first point in a fluid flow direction at an angle in a range of 90° to 180°.

For example, the eddy generation structure 111 may be a reverse-Tesla structure.

Through the fluid inlet 101, the fluid flows into the microfluidic chip 100 according to an embodiment of the present disclosure. Through the fluid outlet 102, the fluid flow out of the microfluidic chip 100 according to an embodiment of the present disclosure. The fluid inlet 101 may include at least one fluid inlet. The fluid outlet 102 may include at least one fluid outlet. In one embodiment, the microfluidic chip 100 according to the present disclosure may include a plurality of the fluid inlets 101. In this case, at least two fluids may flow into the microfluidic chip 100 according to an embodiment of the present disclosure through the plurality of the fluid inlets 101.

The flow channel 110 connects the fluid inlet 101 and the fluid outlet 102 to each other. Along and in the flow channel, the fluid flows through the microfluidic chip 100 according to an embodiment of the present disclosure.

The eddy generation structure 111 is included in the flow channel 110, and has a structure in which an eddy is generated as the fluid flows within the flow channel 110. The eddy generation structure 111 has the main flow channel 111M and at least one auxiliary flow channel 111S. The auxiliary flow channel 111S branches from the main flow channel 111M and returns to and merges with the main flow channel 111M at an angle oriented so as to at least partially disrupt the flow of the fluid in the main flow channel 111M. At the merging point, the eddy may be generated via collision between the fluid flowing in the main flow channel 111M and the fluid flowing in the auxiliary flow channel 111S.

As described above, the eddy may be generated within the flow channel 110 of the microfluidic chip 100 according to an embodiment of the present disclosure, thereby allowing input fluids to be mixed with each other. As long as the mixing is performed to produce beneficial effects, the type of the fluid entering the microfluidic chip 100 or the type of reaction taking place therein is not particularly limited. In one embodiment, in the microfluidic chip 100, a loop-mediated isothermal amplification (LAMP) reaction may occur within the flow channel 110.

As long as the function as described above is performed, a geometric shape of the flow channel 110 or the eddy generation structure 111 is not particularly limited. In one embodiment, the auxiliary flow channel 111S of the eddy generation structure 111 may merge with the main flow channel 111M at an angle of about 130 to 140°. In the context of the present disclosure, when two channels merge with each other, a merge angle may be defined based on the direction of the fluid flow in each of the channels. Therefore, when the channels merge with each other at an angle greater than 90°, the flows of the fluids in the channels meet with each other so as to interfering with and/or colliding with each other. Furthermore, in one embodiment, the main flow channel 111M may have a width of about 80 to 120 μm.

In one example, as long as the auxiliary flow channel 111S of the eddy generation structure 111 branches from the main flow channel 111M, extends by a certain length, and then merges with the main flow channel 111M to generate the eddy, a specific structure thereof is not particularly limited. In one embodiment, the auxiliary flow channel 111S includes a first portion branching from the first point of the main flow channel 111M and extending by a first length; a second portion extending in a curved manner from an end of the first portion toward the main flow channel 111M; and a third portion extending from an end of the second portion by a second length and joining the second point of the main flow channel 111M. As described above, the second portion of the auxiliary flow channel 111S is curved toward the main flow channel 111M, so that, as described above, the auxiliary flow channel 111S merges with the main flow channel 111M at an angle of about 130 to 140°.

In one embodiment, the auxiliary flow channels 111S may be disposed on both opposing sides of the main flow channel 111M and may be arranged in a zigzag manner along the main flow channel 111M. As a result, the eddy generation structure 111 may be formed relatively space-efficiently. In one embodiment, the main flow channel 111M may be formed in a serpentine structure including a plurality of rows.

As described above, the microfluidic chip 100 according to an embodiment of the present disclosure may mix the plurality of fluids input thereto using the generated eddy. Accordingly, the plurality of fluids may be injected into the microfluidic chip 100 through the plurality of fluid inlets 101, respectively. In one embodiment, the microfluidic chip 100 may include the plurality of fluid inlets 101.

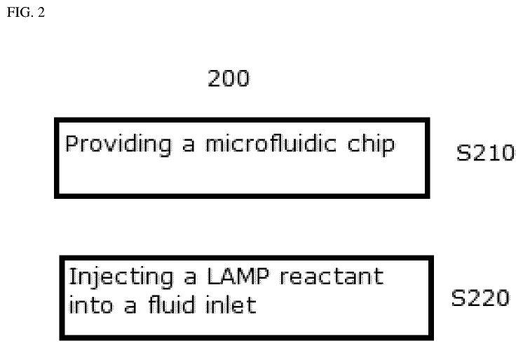

FIG. 2 is a flowchart showing the LAMP gene amplification method according to an embodiment of the present disclosure.

Referring to FIG. 2, a LAMP gene amplification method 200 according to an embodiment of the present disclosure may include providing a microfluidic chip 100 in S210, and injecting a LAMP reactant into a fluid channel of the microfluidic chip 100 such that the LAMP reactant flows through and along a flow channel thereof in S220.

In S210, the microfluidic chip 100 is provided.

Referring to FIG. 1, a microfluidic chip 100 according to an embodiment of the present disclosure includes a fluid inlet 101; a fluid outlet 102; and a flow channel 110 that connects the fluid inlet 101 and the fluid outlet 102 to each other and allows fluid to flow therein, wherein the flow channel 110 includes a plurality of eddy generation structures 111 connected in series with each other, and each of the eddy generation structures 111 includes a main flow channel 111M, and one or more auxiliary flow channels 111S branched from the main flow channel 111M at a first point thereof, and merges with the main flow channel 111M at a second point thereof spaced apart from the first point in a fluid flow direction at an angle in a range of 90° to 180°.

For example, the eddy generation structure 111 may be a reverse-Tesla structure.

Through the fluid inlet 101, the fluid flows into the microfluidic chip 100 according to an embodiment of the present disclosure. Through the fluid outlet 102, the fluid flow out of the microfluidic chip 100 according to an embodiment of the present disclosure. The fluid inlet 101 may include at least one fluid inlet. The fluid outlet 102 may include at least one fluid outlet. In one embodiment, the microfluidic chip 100 according to the present disclosure may include a plurality of the fluid inlets 101. In this case, at least two fluids may flow into the microfluidic chip 100 according to an embodiment of the present disclosure through the plurality of the fluid inlets 101.

The flow channel 110 connects the fluid inlet 101 and the fluid outlet 102 to each other. Along and in the flow channel, the fluid flows through the microfluidic chip 100 according to an embodiment of the present disclosure.

The eddy generation structure 111 is included in the flow channel 110, and has a structure in which an eddy is generated as the fluid flows within the flow channel 110. The eddy generation structure 111 has the main flow channel 111M and at least one auxiliary flow channel 111S. The auxiliary flow channel 111S branches from the main flow channel 111M and returns to and merges with the main flow channel 111M at an angle oriented so as to at least partially disrupt the flow of the fluid in the main flow channel 111M. At the merging point, the eddy may be generated via collision between the fluid flowing in the main flow channel 111M and the fluid flowing in the auxiliary flow channel 111S.

As described above, the eddy may be generated within the flow channel 110 of the microfluidic chip 100 according to an embodiment of the present disclosure, thereby allowing input fluids to be mixed with each other. As long as the mixing is performed to produce beneficial effects, the type of the fluid entering the microfluidic chip 100 or the type of reaction taking place therein is not particularly limited. In one embodiment, in the microfluidic chip 100, a loop-mediated isothermal amplification (LAMP) reaction may occur within the flow channel 110.

As long as the function as described above is performed, a geometric shape of the flow channel 110 or the eddy generation structure 111 is not particularly limited. In one embodiment, the auxiliary flow channel 111S of the eddy generation structure 111 may merge with the main flow channel 111M at an angle of about 130 to 140°. In the context of the present disclosure, when two channels merge with each other, a merge angle may be defined based on the direction of the fluid flow in each of the channels. Therefore, when the channels merge with each other at an angle greater than 90°, the flows of the fluids in the channels meet with each other so as to interfering with and/or colliding with each other. Furthermore, in one embodiment, the main flow channel 111M may have a width of about 80 to 120 μm.

In one example, as long as the auxiliary flow channel 111S of the eddy generation structure 111 branches from the main flow channel 111M, extends by a certain length, and then merges with the main flow channel 111M to generate the eddy, a specific structure thereof is not particularly limited. In one embodiment, the auxiliary flow channel 111S includes a first portion branching from the first point of the main flow channel 111M and extending by a first length; a second portion extending in a curved manner from an end of the first portion toward the main flow channel 111M; and a third portion extending from an end of the second portion by a second length and joining the second point of the main flow channel 111M. As described above, the second portion of the auxiliary flow channel 111S is curved toward the main flow channel 111M, so that, as described above, the auxiliary flow channel 111S merges with the main flow channel 111M at an angle of about 130 to 140°.

In one embodiment, the auxiliary flow channels 111S may be disposed on both opposing sides of the main flow channel 111M and may be arranged in a zigzag manner along the main flow channel 111M. As a result, the eddy generation structure 111 may be formed relatively space-efficiently. In one embodiment, the main flow channel 111M may be formed in a serpentine structure including a plurality of rows.

As described above, the microfluidic chip 100 according to an embodiment of the present disclosure may mix the plurality of fluids input thereto using the generated eddy. Accordingly, the plurality of fluids may be injected into the microfluidic chip 100 through the plurality of fluid inlets 101, respectively. In one embodiment, the microfluidic chip 100 may include the plurality of fluid inlets 101.

In S220, the LAMP reactant is input to the fluid inlet and flows through and along the flow channel of the microfluidic chip 100.

Hereinafter, examples of the present disclosure are described in detail. However, the examples as described below are only some embodiments of the present disclosure, and the scope of the present disclosure is not limited to the examples below.

### Manufacturing Example

A microfluidic chip (Comparative Example) including a serpentine structure and a microfluidic chip (Present Example) of the present disclosure including the serpentine structure and the eddy generation structure were manufactured. To achieve a hydrophilic surface, each of the microfluidic chips was treated with oxygen plasma.

### Method Example

RNA and LAMP reaction reagent were injected into each of the microfluidic chip according to Comparative Example and the microfluidic chip according to Present Example. After the injection, the mixing efficiency of the microfluidic chip according to Comparative Example was measured to be about 70%, and the mixing efficiency of the microfluidic chip according to Present Example was measured to be about 85%.

Although embodiments of the present disclosure have been described with reference to the accompanying drawings, embodiments of the present disclosure are not limited to the above embodiments, but may be implemented in various different forms. A person skilled in the art may appreciate that the present disclosure may be practiced in other concrete forms without changing the technical spirit or essential characteristics of the present disclosure. Therefore, it should be appreciated that the embodiments as described above is not restrictive but illustrative in all respects.

What is claimed is:

1. A microfluidic chip comprising:
a fluid inlet;
a fluid outlet; and
a flow channel constructed to connect the fluid inlet and the fluid outlet to each other, wherein a fluid flows in the flow channel,
wherein the flow channel includes a plurality of eddy generation structures connected in series with each other,
wherein each of the eddy generation structures includes:
a main flow channel; and
at least one auxiliary flow channel branching from the main flow channel at a first point thereof, and merging with the main flow channel at a second point thereof spaced apart from the first point in a fluid flow direction, wherein the auxiliary flow channel meets with the main flow channel at an angle in a range of 90° exclusive to 180° exclusive.

2. The microfluidic chip of claim 1, wherein a loop-mediated isothermal amplification (LAMP) reaction is carried out within the flow channel.

3. The microfluidic chip of claim 1, wherein the auxiliary flow channel of the eddy generation structure meets with the main flow channel at an angle of 130 to 140°.

4. The microfluidic chip of claim 1, wherein the main flow channel has a width of 80 to 120 μm.

5. The microfluidic chip of claim 1, wherein an auxiliary flow channel includes:
a first portion branching from the first point of the main flow channel and extending by a first length;
a second portion extending in a curved manner from an end of the first portion toward the main flow channel; and
a third portion extending from an end of the second portion by a second length and merging with the second point of the main flow channel.

6. The microfluidic chip of claim 1, wherein the at least one auxiliary flow channel includes a plurality of auxiliary flow channels disposed on both opposing sides of the main flow channel and arranged in a zigzag manner along the main flow channel.

7. The microfluidic chip of claim 6, wherein the main flow channel is formed in a serpentine structure including a plurality of rows.

8. The microfluidic chip of claim 1, wherein the fluid inlet includes a plurality of the fluid inlets.

9. A LAMP gene amplification method comprising:
providing a microfluidic chip, wherein the microfluidic chip includes:
a fluid inlet;
a fluid outlet; and
a flow channel constructed to connect the fluid inlet and the fluid outlet to each other, wherein a fluid flows in the flow channel,
wherein the flow channel includes a plurality of eddy generation structures connected in series with each other,
wherein each of the eddy generation structures includes:
a main flow channel; and
at least one auxiliary flow channel branching from the main flow channel at a first point thereof, and merging with the main flow channel at a second point thereof spaced apart from the first point in a fluid flow direction, wherein the auxiliary flow channel meets with the main flow channel at an angle in a range of 90° exclusive to 180° exclusive; and
injecting a LAMP reactant into the fluid inlet such that the LAMP reactant flows along the flow channel.

10. The method of claim 9, wherein the auxiliary flow channel of the eddy generation structure meets with the main flow channel at an angle of 130 to 140°.

11. The method of claim 9, wherein the main flow channel has a width of 80 to 120 μm.

12. The method of claim 9, wherein an auxiliary flow channel includes:
a first portion branching from the first point of the main flow channel and extending by a first length;
a second portion extending in a curved manner from an end of the first portion toward the main flow channel; and
a third portion extending from an end of the second portion by a second length and merging with the second point of the main flow channel.

13. The method of claim 9, wherein the at least one auxiliary flow channel includes a plurality of auxiliary flow channels disposed on both opposing sides of the main flow channel and arranged in a zigzag manner along the main flow channel.

14. The method of claim 13, wherein the main flow channel is formed in a serpentine structure including a plurality of rows.

15. The method of claim 9, wherein the fluid inlet includes a plurality of the fluid inlets.
